(19) United States
(12) Patent Application Publication
(10) Pub. No.: US 2011/0282209 A1
(43) Pub. Date: Nov. 17, 2011

(54) ULTRASOUND OBSERVATION APPARATUS AND CONTROL METHOD OF ULTRASOUND OBSERVATION APPARATUS

(75) Inventors: Hironaka MIYAKI, Tokyo (JP); Takahiro ECHIZENYA, Tokyo (JP); Shuji OTANI, Tokyo (JP)

(73) Assignee: OLYMPUS MEDICAL SYSTEMS CORP., Tokyo (JP)

(21) Appl. No.: 13/046,104

(22) Filed: Mar. 11, 2011

Related U.S. Application Data
(63) Continuation of application No. PCT/JP2010/067950, filed on Oct. 13, 2010.

(30) Foreign Application Priority Data
Nov. 16, 2009 (JP) 2009-261153

(51) Int. Cl. A61B 8/12

(52) U.S. Cl. 600/447

(57) ABSTRACT

An ultrasound observation apparatus of the invention includes: an ultrasound probe portion capable of two-dimensional scanning of an ultrasound beam; a transmission/reception control section for controlling a transmission/reception direction of the ultrasound beam by the ultrasound probe portion; a B-mode image calculation section for generating a B-mode image on a scanning plane based on a result of scanning of the ultrasound beam; a storage section for storing a predetermined sample image determined according to a shape of a treatment instrument in a case where a central axis of the treatment instrument for performing a treatment on a subject and the scanning plane agree with each other; and a correlation calculation section for calculating a correlation value between the B-mode image and the sample image, wherein the transmission/reception control section moves the scanning plane such that the correlation value becomes the maximum.

### CROSS REFERENCE TO RELATED APPLICATION

This application is a continuation application of PCT/JP2010/067950 filed on Oct. 13, 2010 and claims benefit of Japanese Application No. 2009-261153 filed in Japan on Nov. 16, 2009, the entire contents of which are incorporated herein by this reference.

### BACKGROUND OF THE INVENTION

1. Field of the Invention

The present invention relates to an ultrasound observation apparatus and a control method of the ultrasound observation apparatus for generating a B-mode image of inside a subject by scanning an ultrasound beam on a predetermined scanning plane in the subject.

2. Description of the Related Art

As disclosed in a patent document 1, for example, an ultrasound observation apparatus used in a medical field and the like is provided with an ultrasound probe capable of transmitting and receiving ultrasound to and from a subject and generates a B-mode image as a cross-sectional image of a subject. The B-mode image is acquired by scanning an ultrasound beam on a predetermined scanning plane.

As disclosed in the Japanese Patent Application Laid-Open Publication No. 2006-175006, using such an ultrasound observation apparatus makes it possible to perform a treatment while checking on a B-mode image a position and a posture of a treatment instrument such as a puncturing needle, a biopsy forceps, or cytological brush with respect to a predetermined region in a subject.

### SUMMARY OF THE INVENTION

According to one aspect of the present invention, it is possible to provide an ultrasound observation apparatus for generating a B-mode image of inside a subject by scanning an ultrasound beam on a scanning plane in the subject, which includes: an ultrasound probe portion capable of two-dimensional scanning of an ultrasound beam by changing a transmission/reception direction of the ultrasound beam in a first direction and in a second direction; a transmission/reception control section for controlling the transmission/reception direction of the ultrasound beam by the ultrasound probe portion; a B-mode image calculation section for generating the B-mode image based on a result of scanning of the ultrasound beam in the first direction; a storage section for storing a sample image determined according to a shape of a treatment instrument in a case where a central axis of the treatment instrument for performing a treatment on the subject and the scanning plane agree with each other; and a correlation calculation section for calculating a correlation value between the B-mode image and the sample image, wherein the transmission/reception control section moves the scanning plane in the second direction such that the correlation value becomes the maximum.

### BRIEF DESCRIPTION OF THE DRAWINGS

FIG. 1 is a view showing a schematic configuration of an ultrasound observation apparatus according to a first embodiment.

FIG. 2 is a perspective view showing a detailed configuration of a distal end portion of an insertion portion of an ultrasound endoscope according to the first embodiment.

FIG. 3 shows a side surface part of the distal end portion of the insertion portion according to the first embodiment.

FIG. 4 is a view of the distal end portion of the insertion portion according to the first embodiment when viewed from a distal end direction of an insertion axis.

FIG. 5 is a view illustrating a configuration of an ultrasound observation control unit according to the first embodiment.

FIG. 6 is a view showing an example of a B-mode image in a case where a treatment instrument exists on a scanning plane according to the first embodiment.

FIG. 7 is a view showing an example of a sample image according to the first embodiment.

FIG. 8 is a flowchart describing an operation of the ultrasound observation control unit according to the first embodiment.

FIG. 9 is a flowchart of a scanning plane optimization process according to the first embodiment.

FIG. 10 is a view showing a state where an advancing direction of the treatment instrument according to the first embodiment is deviated from a central axis of a treatment instrument insertion port.

FIG. 11 is a view showing an example of a B-mode image in a case where the treatment instrument is deviated from the scanning plane according to the first embodiment.

FIG. 12 is a view showing a configuration of an ultrasound probe portion according to a second embodiment.

FIG. 13 is a view showing a configuration of an ultrasound observation control unit according to the second embodiment.

### DETAILED DESCRIPTION OF THE PREFERRED EMBODIMENTS

Hereinafter, preferred embodiments of the present invention will be described with reference to drawings. Note that, in the drawings used for description below, a different scale size is used for each of the components in order to allow each of the components to be illustrated in a recognizable size in the drawings, and the present invention is not limited to the number, the shapes, the ratio of the sizes of the components, and a relative positional relationship among the components shown in these drawings.

### First Embodiment

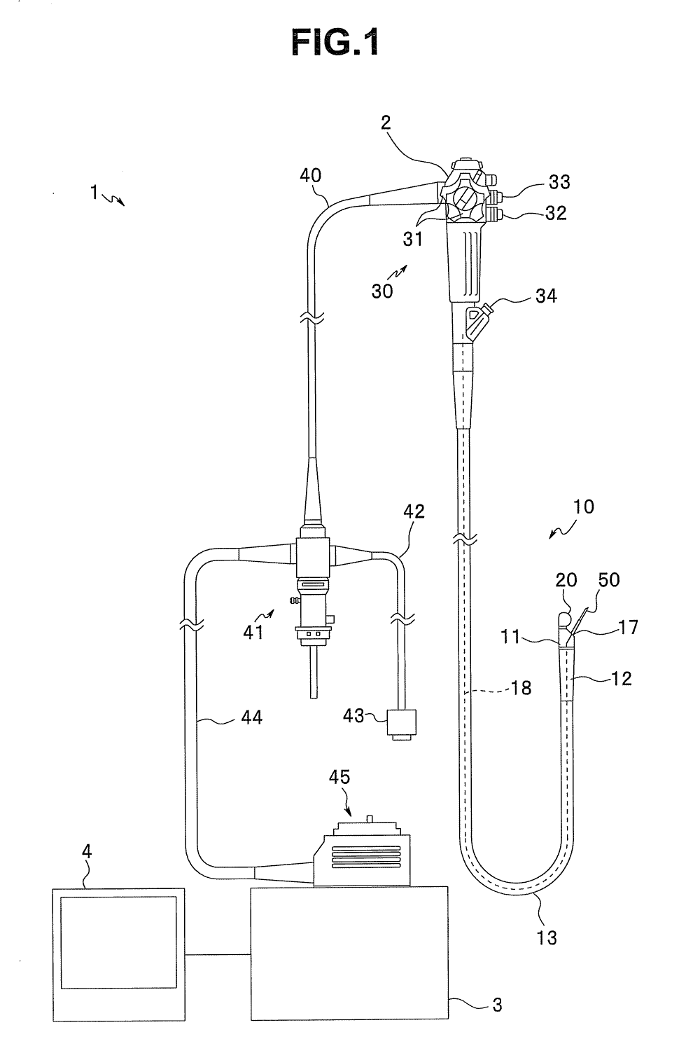

The first embodiment of the present invention will be described below. An ultrasound observation apparatus 1 according to the present embodiment shown in FIG. 1 includes an ultrasound endoscope 2 and an ultrasound observation control unit 3.

The ultrasound observation apparatus 1 is schematically an apparatus which generates a B-mode image (ultrasound tomographic image) of a predetermined region in a subject by scanning an ultrasound beam in the subject, to output the B-mode image to an image display apparatus 4.

The ultrasound endoscope 1 is configured by mainly including an insertion portion 10 which can be introduced in the subject, an operation portion 30 positioned at a proximal end of the insertion portion 10, a universal cord 40 which extends from a side portion of the operation portion 30.

The insertion portion 10 includes the following components in a linked manner: a distal end portion 11 disposed at a distal end of the insertion portion; a bendable bending portion 12 disposed at a proximal end side of the distal end portion 11, and a flexible tube portion 13 having flexibility which is disposed at a proximal end side of the bending portion 12 and connected to a distal end side of the operation portion 30. Though detailed description will be made later, the distal end portion 11 is provided with an ultrasound probe portion 20, a treatment instrument insertion port 17, a fluid feeding portion 14, an image pickup apparatus 15, an illumination device 16, and the like.

The insertion portion 10A includes inside thereof a treatment instrument insertion conduit 18. The treatment instrument insertion conduit 18 is a conduit for communicating the treatment instrument insertion port 17 as an opening portion provided at the distal end portion 10 with a conduit cap 34 provided at the operation portion 30.

The operation portion 30 is provided with an angle knob 31 for operating bending of the bending portion 12, an air-feeding/water-feeding button 32 for controlling a feeding operation of fluid from the fluid feeding portion 14 provided at the distal end portion 10, a suction button 33 for controlling a suction operation from the treatment instrument insertion port 17, the conduit cap 34, and the like.

An endoscope connector 41 to be connected to a light source device, not shown, is provided at a proximal end portion of the universal cord 40. The light emitted from the light source device passes through the universal cord 40, the operation portion 30, and an optical fiber cable inserted through the insertion portion 10, to be emitted from the illumination device 16 of the distal end portion 11. Note that the ultrasound endoscope 1 may be provided with a light source device such as an LED at the distal end portion 11.

An electric cable 42 and an ultrasound cable 44 are extended from the endoscope connector 41. The electric cable 42 is detachably connected to a camera control unit, not shown, through an electric connector 43. The camera control unit is electrically connected to the image pickup apparatus 15 provided at the distal end portion 11 through the electric cable 42. The camera control unit is electrically connected to the image display apparatus 4 and outputs an image picked up by the image pickup apparatus 15 to the image display apparatus 4.

The ultrasound cable 44 is detachably connected to the ultrasound observation control unit 3 to be detailed later, through an ultrasound connector 45.

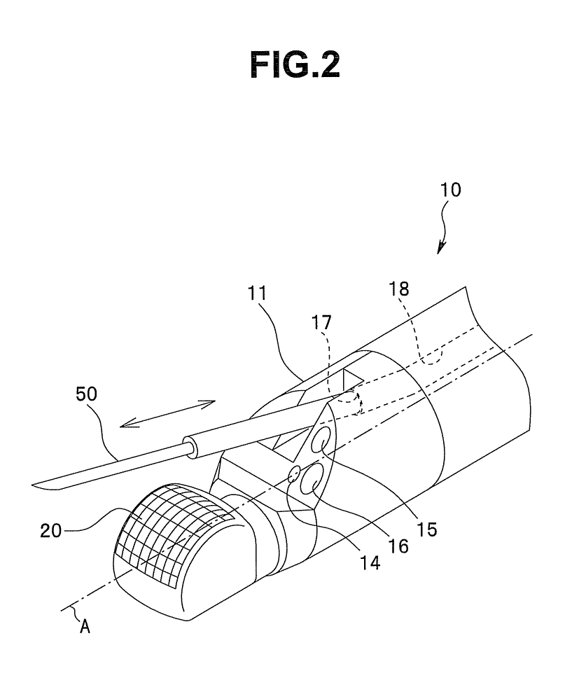

Next, description will be made on a detailed configuration of the distal end portion 11 of the insertion portion 10 of the ultrasound endoscope 2. As shown in FIG. 2, the distal end portion 11 includes the ultrasound probe portion 20, the treatment instrument insertion port 17, the image pickup apparatus 15, the illumination device 16, and the fluid feeding portion 14.

The ultrasound probe portion 20 is configured to be capable of changing the transmission/reception direction of the ultrasound beam in a first direction L1 and in a second direction L2. That is, the ultrasound probe portion 20 is configured so as to enable two-dimensional scanning of an ultrasound beam.

The configuration of the ultrasound probe portion 20 is not specifically limited as long as the two-dimensional scanning of the ultrasound beam is possible. In the present embodiment, as an example, the ultrasound probe portion 20 includes a plurality of ultrasound transducers which are aligned in matrix and can be individually driven, and is configured to electronically perform the two-dimensional scanning of the ultrasound beam by controlling a driving timing of each of the ultrasound transducers.

As the ultrasound transducers constituting the ultrasound probe portion 20, piezoelectric elements or electrostrictive elements such as piezoelectric ceramics, ultrasonic transducers by micromachine technology (MUT: Micromachined Ultrasonic Transducer), or the like can be applied, for example.

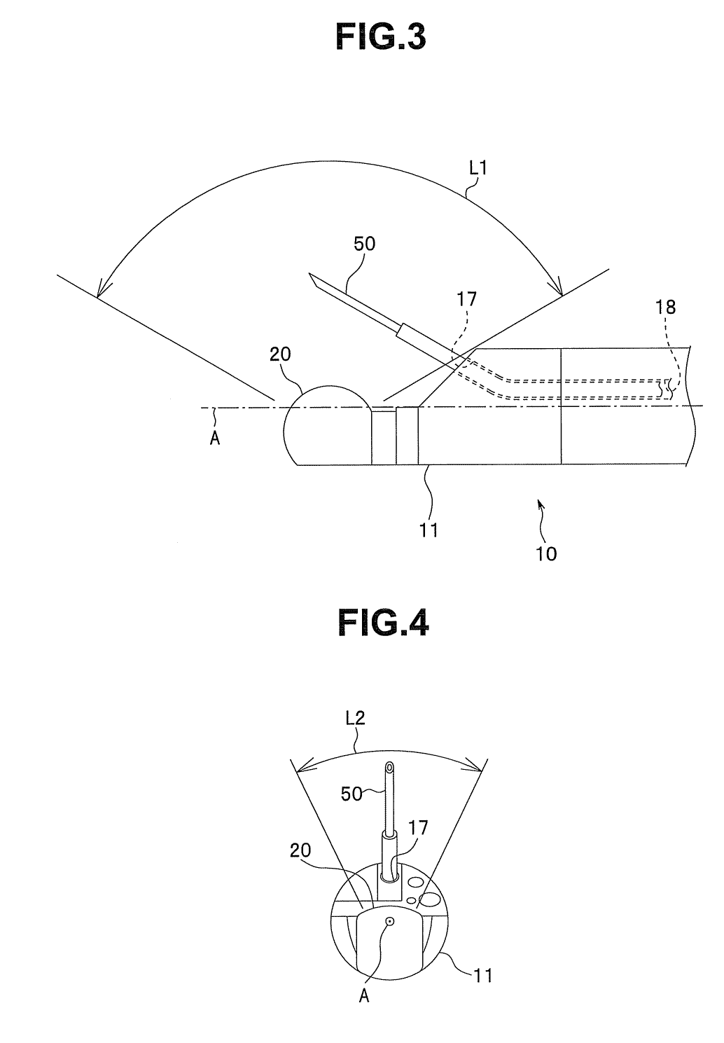

More specifically, the ultrasound probe portion 20 of the present embodiment can change the transmission/reception direction of the ultrasound beam in a substantially sector shape on a plane parallel to an insertion axis A of the insertion portion 10 to perform scanning of an ultrasound beam, as shown in FIG. 3. In the present embodiment, as shown by the arrow L1 in FIG. 3, the amplitude direction of the scanning of the ultrasound beam on the plane substantially parallel to the insertion axis A is defined as a first direction L1.

In addition, in the present embodiment, the plane including a central axis of the ultrasound beam which is scanned in the first direction L1 is referred to as a scanning plane, and the B-mode image generated by the ultrasound observation apparatus 1 is assumed to be acquired by scanning the ultrasound beam on the scanning plane.

As shown in FIG. 4, the ultrasound probe portion 20 of the present embodiment can change the transmission/reception direction of the ultrasound beam in a substantially sector shape on the plane perpendicular to the insertion axis A of the insertion portion 10 to perform scanning of the ultrasound beam. In the present embodiment, as shown by the arrow L2 in FIG. 4, the amplitude direction of the scanning of the ultrasound beam on the plane perpendicular to the insertion axis A is defined as a second direction L2.

That is, the ultrasound probe portion 20 of the present embodiment enables the scanning plane to move in the second direction L2 by changing the transmission/reception direction of the ultrasound beam in the second direction L2. In the present embodiment, as an example, the scanning plane can be moved to positions of a plurality of predetermined locations within the range of scanning in the second direction L2.

Note that the ultrasound probe portion 20 according to the present embodiment which is shown in drawings is configured by aligning the ultrasound transducers in a substantially circular arc shape along the first direction L1 and the second direction L2. However, it is needless to say that electronic two-dimensional scanning of the ultrasound beam is possible even if the plurality of ultrasound transducers are aligned in matrix on a plane.

The treatment instrument insertion port 17 is an opening portion for allowing a treatment instrument 50 to protrude therefrom, and communicates with the treatment instrument insertion conduit 18. In the ultrasound endoscope 2 according to the present embodiment, the treatment instrument 50 is inserted from the opening portion of the conduit cap 34 to allow the treatment instrument 50 to protrude from the treatment instrument insertion port 17 of the distal end portion 11, thereby enabling the treatment instrument 50 to be introduced into a body of a subject, for example.

Note that the type of the treatment instrument 50 is not specifically limited, but a puncture needle, a biopsy forceps, a cytological brush, or the like can be listed, for example. In the present embodiment, the treatment instrument 50 is a puncture needle, as shown in FIG. 2. The treatment instrument 50 may be provided with an ultrasound scattering portion for scattering ultrasound waves in order to make the echo pattern of the treatment instrument 50 in a B-mode image clearer.

Furthermore, in the present embodiment, the treatment instrument insertion port 17 is disposed such that the treatment instrument 50 protruded from the treatment instrument insertion port 17 advances in a range of scanning of the ultrasound beam performed by the ultrasound probe portion 20, as shown in FIGS. 3 and 4.

In other words, the ultrasound observation apparatus 1 according to the present embodiment is configured to be able to capture the treatment instrument 50 protruded from the treatment instrument insertion port 17 in a B-mode image by moving the scanning plane.

In addition, the present embodiment employs, as one example, a configuration in which, when the scanning plane is moved to the center of the range of scanning in the second direction L2, the scanning plane and the central axis of the treatment instrument insertion port 17 are positioned on substantially the same plane.

The image pickup apparatus 15 includes an image-forming optical system member and an image pickup device, and picks up an optical image. The image pickup apparatus 15 is disposed so as to capture the distal end direction along the insertion axis A within the field of view thereof. The illumination device 16 emits the light generated by the light source device into the field of view of the image pickup apparatus 15. The fluid feeding portion 17 is an opening portion provided at the distal end portion 11, and fluid is fed from the fluid feeding portion 17 by operating the air-feeding/water-feeding button 32 provided at the operation portion 30.

Next, description will be made on the detailed configuration of the ultrasound observation control unit 3. The ultrasound observation control unit 3 includes a calculation device, a storage device, an input/output device, a power control device and the like, and is a control apparatus which controls operation of the ultrasound probe portion 20 and generates and outputs a B-mode image based on a predetermined program.

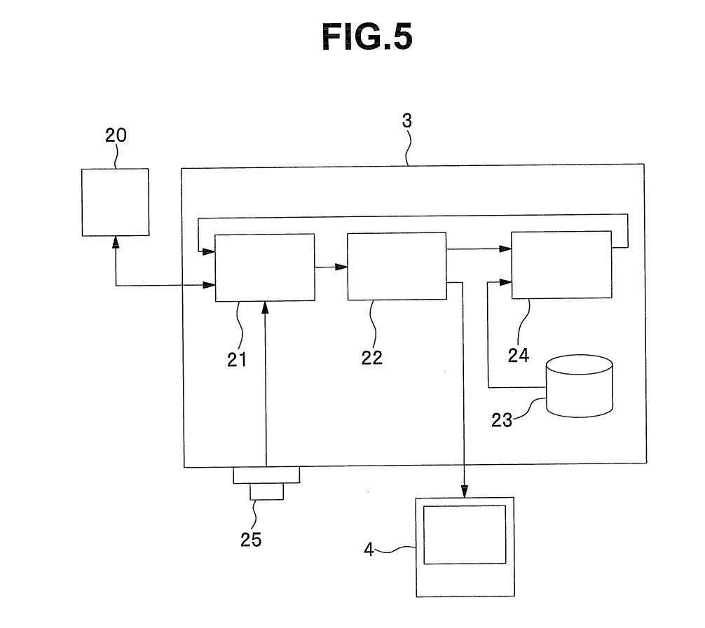

The ultrasound observation control unit 3 includes, as shown in FIG. 5, a transmission/reception control section 21, a B-mode image calculation section 22, a storage section 23, a correlation calculation section 24, and an ultrasound observation switch 25, as components required for achieving the operation to be described later of the ultrasound observation apparatus 1. Note that the transmission/reception control section 21, the B-mode image calculation section 22, and the correlation calculation section 24 may be mounted to the ultrasound observation control unit 3 either in a hardware manner or in a software manner.

The transmission/reception control section 21 controls the transmission/reception direction of the ultrasound beam transmitted and received by the ultrasound probe portion 20. That is, the transmission/reception control section 21 controls the position of the scanning plane with respect to the second direction L2 and the scanning of the ultrasound beam for acquiring the B-mode image on the scanning plane.

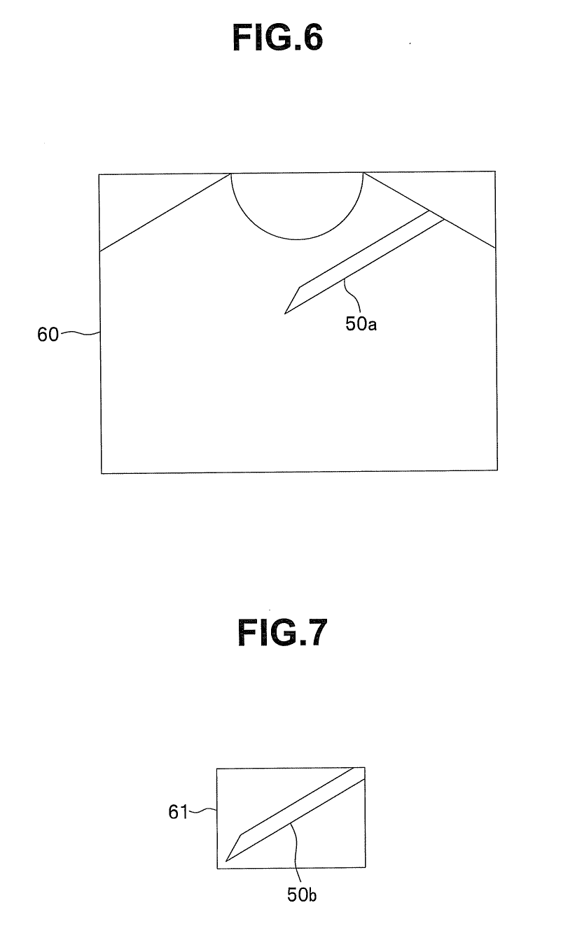

The B-mode image calculation section 22 generates the B-mode image on the scanning plane based on the result of scanning of the ultrasound beam performed by the ultrasound probe portion 20. When the treatment instrument 50 exists on the scanning plane, for example, an echo pattern 50a of the treatment instrument 50 appears in a B-mode image 60, as shown in FIG. 6.

The storage section 23 stores a predetermined sample image determined according to the shape of treatment instrument 50. Specifically, the sample image is an image showing a shape and a size of an ideal echo pattern of the treatment instrument 50 in the B-mode image in the case where the scanning plane and the central axis of the treatment instrument 50 agree with each other.

When the treatment instrument 50 is a puncture needle as in the present embodiment, a sample image 61 is an image showing a shape and a size of an echo pattern 50b of the distal end portion of the puncture needle in the case where the central axis of the puncture needle agrees with the scanning plane, as shown in FIG. 7.

The present embodiment describes, as an example, that the sample image 61 is created in advance according to the type and the shape of the treatment instrument 50. Note that the sample image 61 may be stored in the storage section 23 as a result of designation of the echo pattern 50a of the treatment instrument 50 in the actual B-mode image 60 by a user of the ultrasound observation apparatus 1, that is, may be stored as the echo pattern 50a of the treatment instrument 50 to be actually used, by what is called a teaching operation.

In addition, the shape of the treatment instrument 50 is hard to appear in the B-mode image 60 in some cases depending on a shape of the treatment instrument 50 or a material configuring the treatment instrument 50. However, in such a case, the ultrasound scattering portion is provided in a part of the treatment instrument 50. When the ultrasound scattering portion is provided in the treatment instrument 50, the sample image 61 is an image showing a shape of an ideal echo pattern of the ultrasound scattering portion in the B-mode image 60.

The correlation calculation section 24 calculates a correlation value R between the B-mode image 60 and the sample image 61. Specifically, the correlation calculation section 24 performs on the B-mode image 60 an image processing referred to as a pattern matching by using the sample image 61 as a template, and calculates a similarity between the echo pattern in the B-mode image 60 and the sample image 61. The higher the similarity between the echo pattern in the B-mode image 60 and the sample image 61, the higher the correlation value R. Since the pattern matching is a well-known technology, detailed description thereof will be omitted.

The ultrasound observation switch 25 is an input device through which a user inputs instructions for starting and terminating the observation using a B-mode image. In the present embodiment, the ultrasound observation switch 25 is provided to the ultrasound observation control unit 3, as an example. However, the ultrasound observation switch 25 may be provided to the operation portion 30 of the ultrasound endoscope 2, or may be configured as a switch like a foot switch provided separately from the ultrasound observation control unit 3 and the ultrasound endoscope 2.

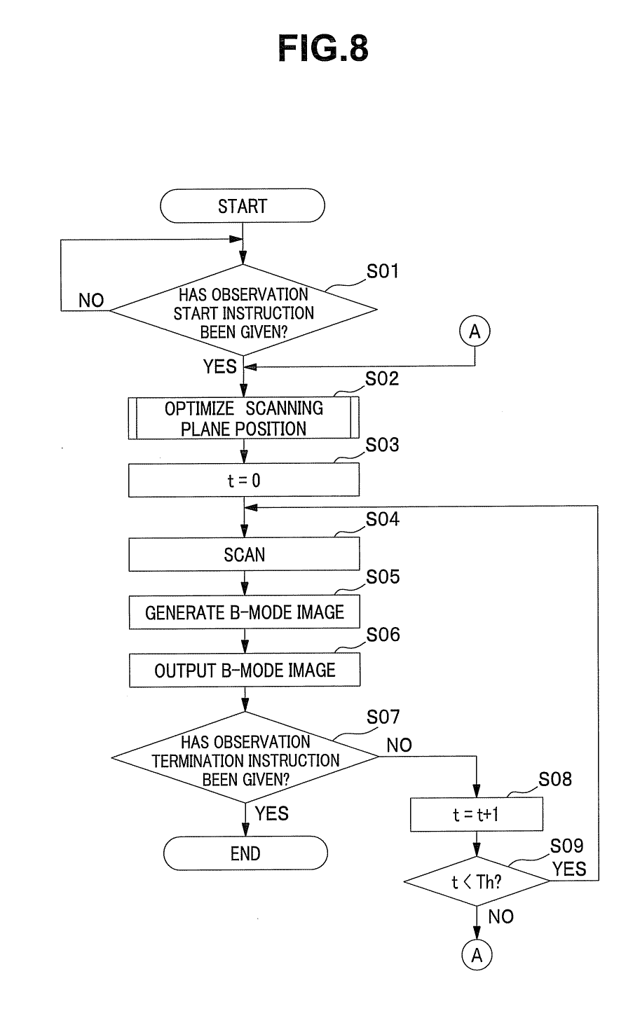

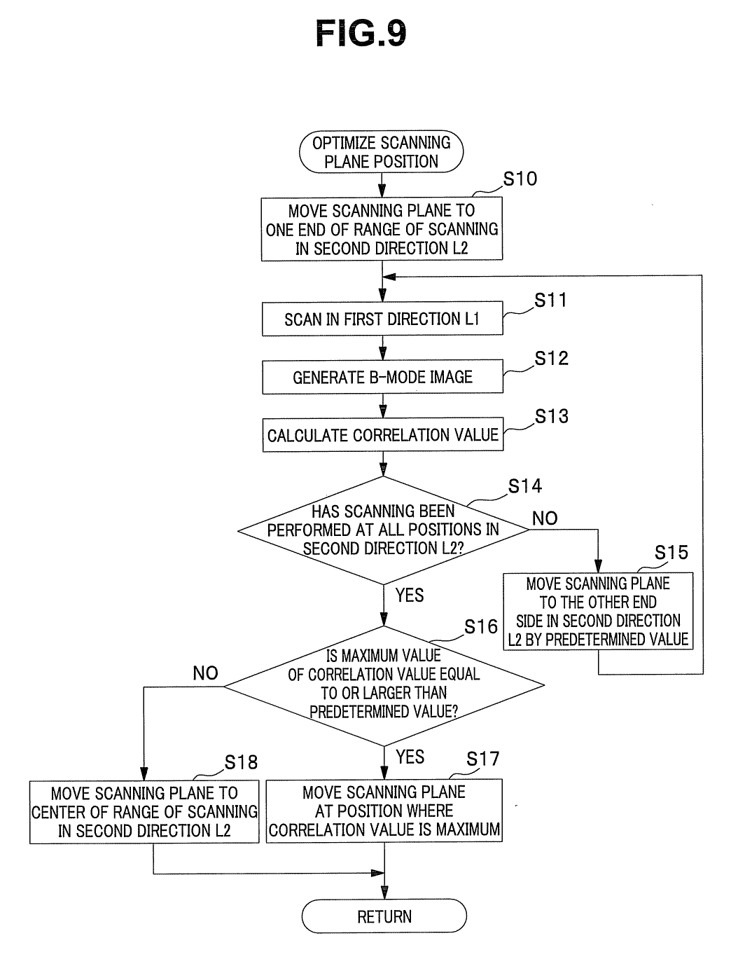

Next, the operation of the ultrasound observation apparatus 1 will be described with reference to the flowcharts in FIGS. 8 and 9. Note that description will be made below on the B-mode image generation operation performed by the ultrasound probe portion 20 and the ultrasound observation control unit 3, and description on the optical image observation operation performed by the image pickup apparatus 15 provided in the ultrasound endoscope 1 will be omitted.

First, in step S01, a stand-by state continues until an instruction for starting the observation using a B-mode image is inputted by operating the ultrasound observation switch 25. When the instruction for starting the observation using a B-mode image is inputted, the process moves to step S02, and the scanning plane position optimization process shown in the flowchart in FIG. 9 is performed.

In the scanning plane position optimization process, first in step S10, the transmission/reception control section 21 moves the scanning plane to one end of the range of scanning in the second direction L2. Then, in step S11, the transmission/reception control section 21 controls the ultrasound probe portion 20 to cause the ultrasound beam to scan in the first direction L1 on the scanning plane whose position with respect to the second direction L2 is determined.

Next, in step S12, the B-mode image calculation section 22 generates a B-mode image based on the result of scanning in the step S11. Then, in step S13, the correlation calculation section 24 calculates a correlation value between the B-mode image acquired in the step S12 and the sample image stored in the storage section 23.

Next, in step S14, it is determined whether or not scanning has been performed at all positions determined in advance, regarding the position of the scanning plane with respect to the second direction L2. When scanning has not been performed at all the positions, regarding the second direction L2, the process moves to step S15. In the step S15, after the scanning plane is moved to the next position on the other end side in the second direction L2, the processes in the step S11 to step S13 are repeated.

Then, in the step S14, when it is determined that scanning has been performed at all the positions determined in advance regarding the position of the scanning plane with respect to the second direction L2, the process moves to step S16.

That is, the steps S10 to S15 are the processes of acquiring a plurality of B-mode images with the scanning plane positioned at a plurality of locations in the second direction L2, and calculating correlation values between the plurality of B-mode images and the sample image.

In the step S16, it is determined whether or not the maximum value of the correlation values between the plurality of B-mode images acquired in the above process and the sample image is equal to or larger than a predetermined threshold. When the maximum value of the correlation values is equal to or larger than the predetermined threshold, the process moves to step S17.

In step S17, the transmission/reception control section 21 moves the position of the scanning plane with respect to the second direction L2 to a position where the maximum correlation value is acquired among the correlation values of the plurality of B-mode images. That is, the step S17 is a process of moving the scanning plane such that the correlation value between the B-mode image and the sample image becomes the maximum.

On the other hand, when the maximum value of the correlation values is smaller than the predetermined threshold, the process moves to step S18. In the step S18, the transmission/reception control section 21 moves the position of the scanning plane with respect to the second direction L2 to the center of the range of scanning. The state where the maximum value of the correlation values is not equal to or larger than the predetermined threshold is supposed to be the state where the treatment instrument 50 is not protruded from the treatment instrument insertion port 17. Therefore, in the step S18, the scanning plane is moved to a position where the scanning plane and the central axis of the treatment instrument insertion port 17 substantially agree with each other.

Then, the scanning plane position optimization process is terminated, and the process returns to the step S03 in FIG. 8. In the step S03, the counter value t is reset to be zero (t=0). Then, in the step S04, an ultrasound beam is scanned in the first direction L1 on the scanning plane whose position has been determined in the scanning plane position optimization process in the step S02.

Next, in the step S05, the B-mode image calculation section 22 generates a B-mode image based on the result of the scanning in the step S04. Then, in the step S06, the generated B-mode image is outputted to the image display apparatus 4. According to the process, the B-mode image is displayed on the image display apparatus 4.

Next, in the step S07, it is determined whether or not an instruction for terminating the observation using the B-mode image has been inputted by operating the ultrasound observation switch 25. When the instruction for terminating the observation using the B-mode image has been inputted, the operation is stopped.

On the other hand, when the instruction for terminating the observation using B-mode image is not inputted, the process moves to the step S08, and one is added to the counter value t (t=t+1). Then, in the step S09, it is determined whether or not the counter value t is smaller than a predetermined threshold Th.

When the counter value t is smaller than the predetermined threshold Th, the process returns to step S04, and the scanning of ultrasound beam and the generation of B-mode image are repeated. When the counter value t reaches the predetermined Th, the process returns to the step S02, and the scanning plane position optimization process is performed.

That is, in the present embodiment, after the scanning of the ultrasound beam and the generation of the B-mode image are repeated by a predetermined number of times Th on the scanning plane located at a certain position with respect to the second direction L2, the scanning plane position optimization process is performed.

In the scanning plane position optimization process, the position of the scanning plane is determined such that the correlation value between the B-mode image and the sample image becomes the maximum, that is, the shape of the treatment instrument 50 appears most clearly on the B-mode image.

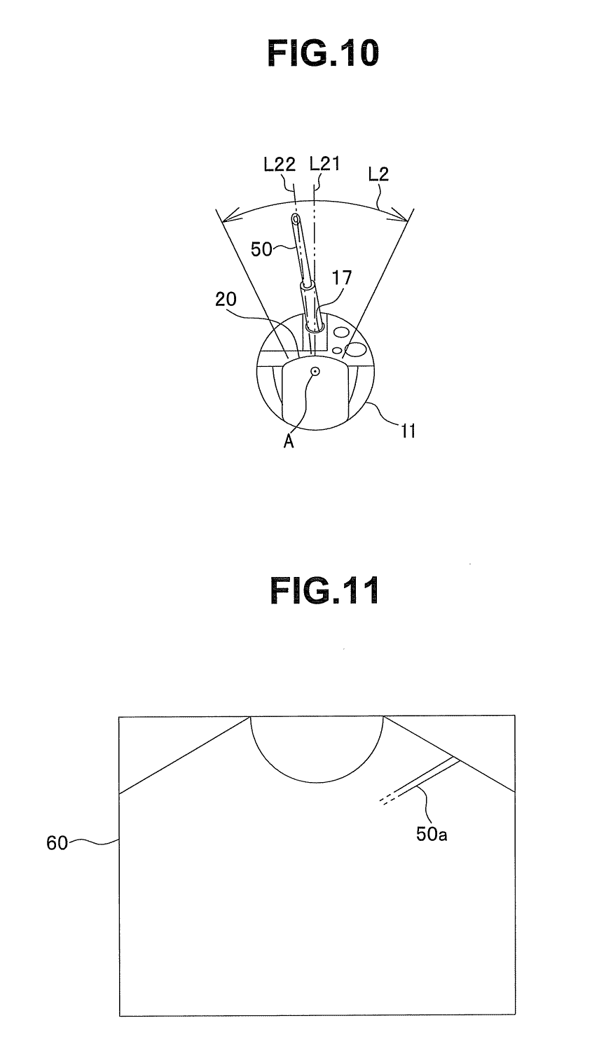

For example, as shown in FIG. 10, in a case where the protruding direction of the treatment instrument 50 protruded from the treatment instrument insertion port 17 is inclined with respect to the central axis of the treatment instrument insertion port 17, if the scanning plane is remained fixed at a position (position shown by the two-dot-chain line L21 in FIG. 10) where the scanning plane substantially agrees with the central axis of the treatment instrument insertion port 17 as in a conventional configuration, observation of the treatment instrument 50 on the B-mode image 60 becomes difficult as shown in FIG. 11.

According to the ultrasound observation apparatus 1 in the present embodiment, even in the case where the protruding direction of the treatment instrument 50 is thus inclined, the scanning plane position optimization process is performed, and thereby the scanning plane is automatically moved to the position (position shown by the two-dot-chain line L22 in FIG. 10) where the echo pattern of the treatment instrument 50 shows the shape of treatment instrument 50 most clearly.

In addition, the scanning plane position optimization process is periodically performed in a period during which the observation using the B-mode image is continued. Therefore, even if the treatment instrument 50 is deviated from the scanning plane in the middle of the treatment performed on a subject, the scanning plane automatically moves, which allows the treatment instrument 50 to be captured again in the B-mode image 60 without the need for the user to intentionally perform any operation.

That is, according to the ultrasound observation apparatus 1 of the present embodiment, it is possible to continue excellent observation of the treatment instrument 50 on the B-mode image 60 without performing any cumbersome operation.

### Second Embodiment

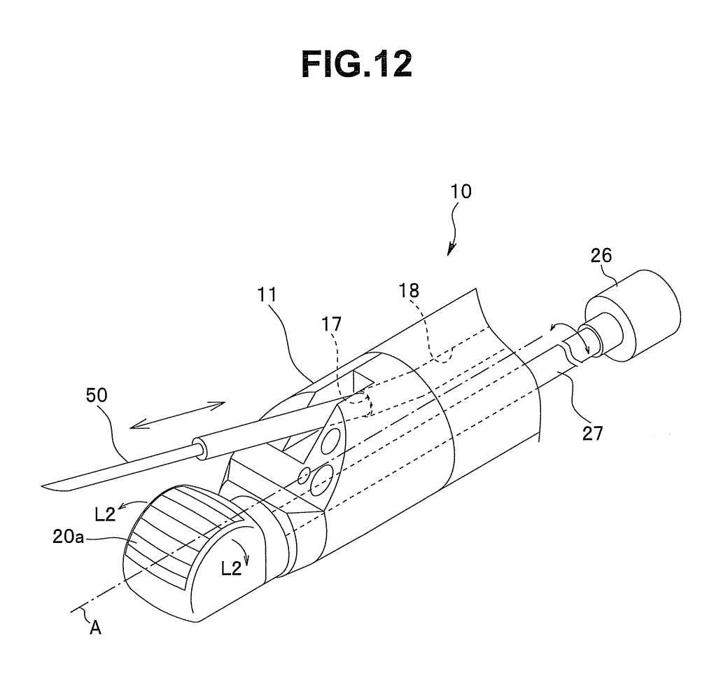

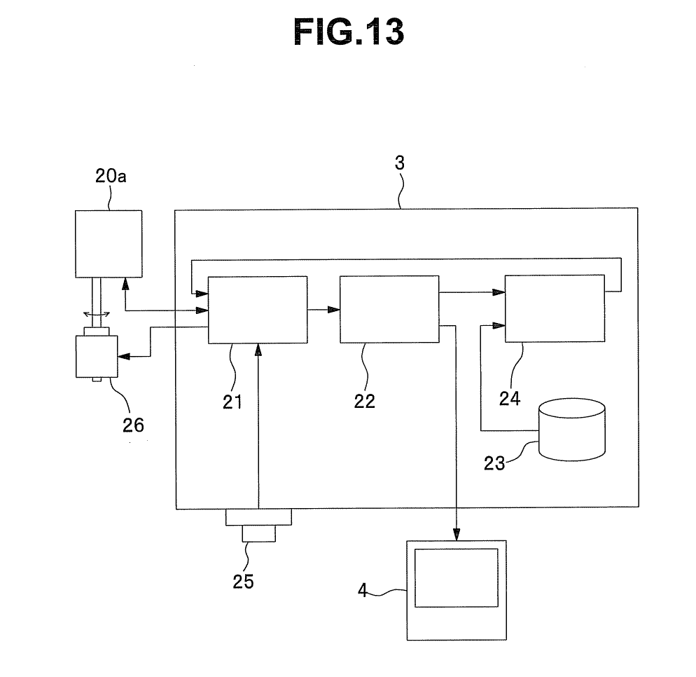

The second embodiment of the present invention will be described below with reference to FIGS. 12 and 13. The ultrasound observation apparatus according to the present embodiment is different from the first embodiment in the method of scanning of ultrasound beam performed by the ultrasound probe portion 20. Only the points different from the first embodiment will be described below, and the same components as those in the first embodiment are attached with the same reference numerals and description thereof will be omitted accordingly.

As shown in FIG. 12, the ultrasound probe portion 20a of the present embodiment has a configuration of what is called a convex scanning type, in which a plurality of ultrasound transducers are aligned in a row in a substantially circular arc shape. A plurality of ultrasound transducers constituting the ultrasound probe portion 20a are aligned such that scanning of an ultrasound beam can be performed in substantially sector shape on the plane parallel to the insertion axis A of the insertion portion 10. That is, the ultrasound probe portion 20a is configured to enable electronic scanning of an ultrasound beam in the first direction L1.

In addition, the ultrasound probe portion 20a is disposed so as to be oscillatable around the axis parallel to the insertion axis A of the insertion portion 10. That is, the ultrasound probe portion 20a is configured to enable mechanical scanning of an ultrasound beam in the second direction L2 by oscillating in the second direction L2.

The ultrasound probe portion 20a is connected to an electric motor 26 provided in the operation portion 30 via a flexible shaft 27 inserted through the insertion portion 10. The ultrasound probe portion 20a oscillates around the axis parallel to the insertion axis A by the drive force generated by the electric motor 26. As shown in FIG. 13, the electric motor 26 is electrically connected to the transmission/reception control section 21, and the operation of the electric motor 26 is controlled by the transmission/reception control section 21.

As described above, the ultrasound probe portion 20a according to the present embodiment can perform two-dimensional scanning of the ultrasound beam by combining the electronic scanning and the mechanical scanning. In addition, the direction of the scanning of the ultrasound beam performed by the ultrasound probe portion 20a is controlled by the transmission/reception control section similarly as in the first embodiment.

It is needless to say that the same effects as those in the first embodiment can be obtained also by the present embodiment described above.

According to the above-described embodiment, it is possible to provide an ultrasound observation apparatus for observing a situation of a treatment using a treatment instrument in a subject, which is capable of continuing excellent observation of the treatment instrument.

The present invention is not limited to the above-described embodiments, and can be modified as needed without departing from the gist of invention or the spirit of invention which can be read from claims and the whole specification. An ultrasound observation apparatus modified as such is also included in the technical range of the present invention.

As described above, the present invention is suitable for an ultrasound observation apparatus which observes a situation of a treatment using a treatment instrument in a subject.

What is claimed is:

1. An ultrasound observation apparatus for generating a B-mode image of inside a subject by scanning an ultrasound beam on a scanning plane in the subject, the ultrasound observation apparatus comprising:
an ultrasound probe portion capable of two-dimensional scanning of an ultrasound beam by changing a transmission/reception direction of the ultrasound beam in a first direction and in a second direction;
a transmission/reception control section for controlling the transmission/reception direction of the ultrasound beam by the ultrasound probe portion;
a B-mode image calculation section for generating the B-mode image based on a result of scanning of the ultrasound beam in the first direction;
a storage section for storing a sample image determined according to a shape of a treatment instrument in a case where a central axis of the treatment instrument for performing a treatment on the subject and the scanning plane agree with each other; and
a correlation calculation section for calculating a correlation value between the B-mode image and the sample image,
wherein the transmission/reception control section moves the scanning plane in the second direction such that the correlation value becomes the maximum.

2. The ultrasound observation apparatus according to claim 1, wherein
the ultrasound probe portion is provided to an ultrasound endoscope which can be introduced into a subject, and
the ultrasound endoscope includes a treatment instrument insertion port for allowing the treatment instrument to protrude into a range of the scanning of the ultrasound beam by the ultrasound probe portion.

3. The ultrasound observation apparatus according to claim 2, wherein
the ultrasound probe portion is arranged such that the scanning plane is parallel to a central axis of the treatment instrument insertion port and the second direction is along a plane which is perpendicular to the scanning plane,
the transmission/reception control section moves the scanning plane to a plurality of locations in the second direction with the treatment instrument protruded from the insertion port,
the B-mode image calculation section generates a plurality of B-mode images acquired in a state where the scanning plane is positioned at the plurality of locations in the second direction,
the correlation calculation section calculates correlation values between the plurality of B-mode images and the sample image, and
the transmission/reception control section moves the scanning plane to a position where the maximum correlation value is acquired among the correlation values of the plurality of B-mode images.

4. The ultrasound observation apparatus according to claim 3, wherein,
when the maximum value of the correlation values is smaller than a predetermined threshold, the transmission/reception section moves the scanning plane to a predetermined position in the second direction.

5. The ultrasound observation apparatus according to claim 1, wherein
the transmission/reception control section periodically moves the scanning plane such that the correlation value becomes the maximum.

6. The ultrasound observation apparatus according to claim 5, wherein,
the transmission/reception control section periodically moves the scanning plane after a predetermined number of times of generation of the B-mode image by the B-mode image calculation section and calculation of the correlation value by the correlation calculation section.

7. The ultrasound observation apparatus according to claim 1, wherein
the sample image is an image showing a shape and a size of the treatment instrument in the B-mode image in a case where the scanning plane and the central axis of the treatment instrument agree with each other.

8. The ultrasound observation apparatus according to claim 7, wherein
the sample image is an image stored in the storage section by designating an echo pattern of the treatment instrument in the B-mode image generated by the B-mode image calculation section.

9. The ultrasound observation apparatus according to claim 1, wherein
the ultrasound probe portion includes a plurality of ultrasound transducers which are aligned in matrix and can be driven individually, and the ultrasound probe portion is configured to electronically perform the two-dimensional scanning of the ultrasound beam by controlling a driving timing of each of the ultrasound transducers.

10. The ultrasound observation apparatus according to claim 1, wherein
the ultrasound probe portion includes a plurality of ultrasound transducers arranged in a row in a circular arc shape, and the ultrasound probe portion is configured to perform the two-dimensional scanning of the ultrasound beam by performing electronic scanning of the ultrasound beam in the first direction by controlling a driving timing of each of the ultrasound transducers, and by performing mechanical scanning of the ultrasound beam in the second direction.

11. The ultrasound observation apparatus according to claim 1, wherein
the sample image shows a shape of a puncture needle, a biopsy forceps, or a cytological brush in the B-mode image.

12. A control method of an ultrasound observation apparatus which generates a B-mode image of inside a subject by scanning an ultrasound beam on a scanning plane in the subject, the control method comprising:
performing two-dimensional scanning of an ultrasound beam by changing a transmission/reception direction of the ultrasound beam in a first direction and in a second direction using an ultrasound probe portion;
controlling the transmission/reception direction of the ultrasound beam by the ultrasound probe portion using a transmission/reception control section;
generating the B-mode image based on a result of scanning of the ultrasound beam in the first direction using a B-mode image calculation section;
storing in a storage section a sample image determined according to a shape of a treatment instrument in a case where a central axis of the treatment instrument for performing a treatment on the subject and the scanning plane agree with each other;
calculating a correlation value between the B-mode image and the sample image using a correlation calculation section; and
moving the scanning plane in the second direction such that the correlation value becomes the maximum using the transmission/reception control section.

13. The control method of the ultrasound observation apparatus according to claim 12, wherein,
when the maximum value of the correlation value is smaller than a predetermined threshold, the scanning plane is moved to a predetermined position in the second direction using the transmission/reception control section.

14. The control method of the ultrasound observation apparatus according to claim 12, wherein the scanning plane is periodically moved such that the correlation value becomes the maximum using the transmission/reception control section.

15. The control method of the ultrasound observation apparatus according to claim 14, wherein the scanning plane is periodically moved using the transmission/reception control section after the B-mode image is generated a predetermined number of times using the B-mode image calculation section and the correlation value is calculated using the correlation calculation section.
